(19) United States
(12) Patent Application Publication
(10) Pub. No.: US 2025/0074872 A1
(43) Pub. Date: Mar. 6, 2025

(54) METHOD FOR PREPARING AND PURIFYING MONOMETHYL AURISTATIN E INTERMEDIATE

(71) Applicant: REMEGEN CO., LTD., Yantai, Shandong (CN)

(72) Inventors: MARIE M. ZHU, Yantai, Shandong (CN); Haitao LI, Yantai, Shandong (CN); Xin LIANG, Yantai, Shandong (CN); Xinli LI, Yantai, Shandong (CN); Xiaoqing SUN, Yantai, Shandong (CN); Peng Sun, Yantai, Shandong (CN); Kai XIAO, Yantai, Shandong (CN); Xiaowei LIANG, Yantai, Shandong (CN); Jinguo GAO, Yantai, Shandong (CN)

(21) Appl. No.: 18/725,704

(22) PCT Filed: Apr. 6, 2023
(86) PCT No.: PCT/CN2023/086387

§ 371 Date: Jun. 28, 2024

(30) Foreign Application Priority Data
Apr. 8, 2022 (CN) 202210366476.1

(51) Int. Cl. C07D 207/08

(52) U.S. Cl. CPC C07D 207/08

(57) ABSTRACT

A method for preparing and purifying a monomethyl auristatin E intermediate. The method has the advantages of few steps, simple operation, cheap and easily available raw materials, high safety, a simple purification method and extremely high purity of a purified product, and the yield of the method can be as high as 95%, which not only improves the safety of the production process, but also significantly improves the yield of the product, and reduces the subsequent production cost. Therefore, the method is suitable for industrial popularization.

### FIELD

The present disclosure relates to the field of compound synthesis methods, and more particularly to a method for preparing and purifying a monomethyl auristatin E (MMAE) intermediate.

### BACKGROUND

Monomethyl auristatin E (MMAE), a fully synthetic derivative of auristatins, can effectively inhibit mitosis by inhibiting tubulin polymerization. It is currently widely used as a cytotoxic component (i.e., drug moiety) to synthesize antibody drug conjugates (ADCs) for the treatment of cancer.

At present, a common route for preparing MMAE is shown as follows, wherein compound b involved in step 1 is the main intermediate in the preparation of MMAE.

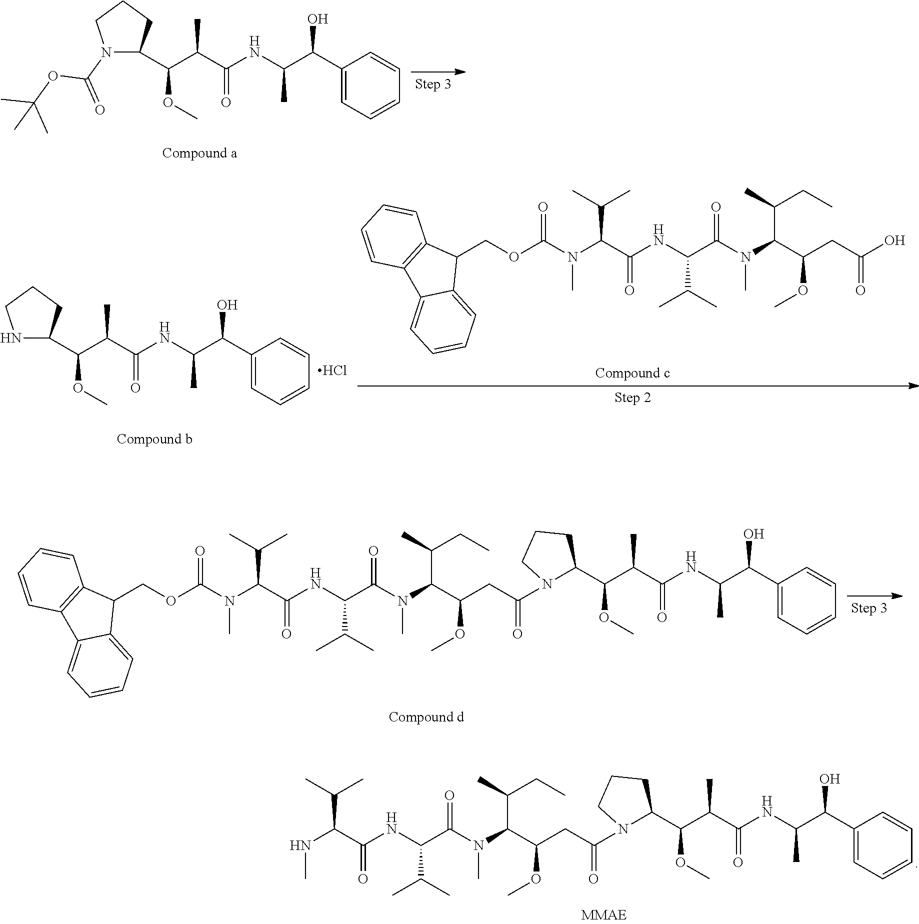

However, the current method for preparing compound b has problems such as low purity or low yield of the prepared product or a large number of residual impurities. For example, the Chinese Patent Application with publication number CN105968038A discloses a method for preparing a dipeptide compound hydrochloride (i.e., compound b), and the route is as follows:

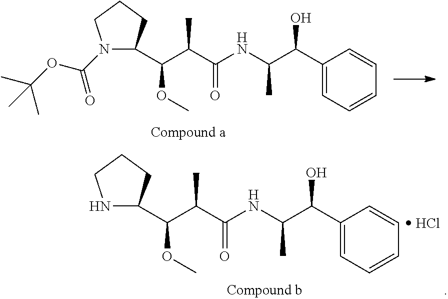 [SMILES: CO[C@H]([C@@H](C)C(=O)N[C@H](C)[C@@H](O)c1ccccc1)[C@@H]1CCCN1C(=O)OC(C)(C)C ; CO[C@@H]([C@@H]1CCCN1)[C@@H](C)C(=O)N[C@H](C)[C@@H](O)c1ccccc1 ; "· HCl"]

In Example 8 on page 7 of the specification of the above application, it discloses a preparation and purification method: after removing the Boc protecting group from compound a by hydrochloric acid method, adding methyl tert-butyl ether, dioxane, and methyl tert-butyl ether in sequence to obtain a crude compound b, and purifying the crude compound b by tetrahydrofuran and heptane to obtain a purified compound b with a purity of 99.6%, but a yield of only 82.3%. In addition, the dioxane involved in this method is very easy to oxidation and is prone to explosion after oxidation, which brings many dangerous factors to safe production.

### SUMMARY

In order to solve the above problems, the present disclosure provides a new method for preparing and purifying a monomethyl auristatin E intermediate, wherein a monomethyl auristatin E intermediate with high purity and low impurity is obtained by removing impurities using an organic reagent with reduced pressure concentration.

Particularly, the monomethyl auristatin E intermediate prepared by the present disclosure has a structure represented by formula (I) (i.e., the above compound b, also referred to as the compound represented by formula (I), the same below):

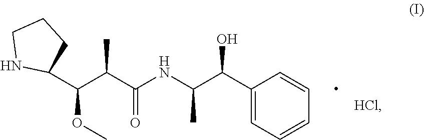 [SMILES: CO[C@@H]([C@@H]1CCCN1)[C@@H](C)C(=O)N[C@H](C)[C@@H](O)c1ccccc1]

the method has a preparation route of:

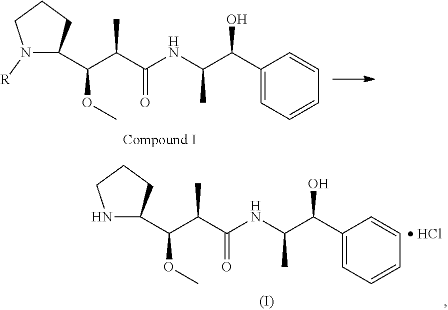

wherein R is an amino protecting group, and the above method comprises the following steps:
A. removing the amino protecting group R from compound 1 by hydrochloric acid method;
B. after completing the reaction of step A, adding an appropriate amount of a first organic solvent to a reaction solution of step A, and concentrating under reduced pressure to obtain a concentrate a;
C. adding an appropriate amount of a second organic solvent to the concentrate a obtained in step B, and concentrating under reduced pressure to obtain a concentrate b; and
D. further purifying the concentrate b,

wherein it can be understood that R can be any amino protecting group that can be removed by hydrochloric acid method. In some preferred embodiments, the amino protecting group R is selected from the group consisting of Boc protecting group, Cbz protecting group, Tfa protecting group, Tos protecting group, Trt protecting group, and DMB protecting group, that is, the structure of the amino protecting group R can be selected from, but not limited to, the group consisting of:

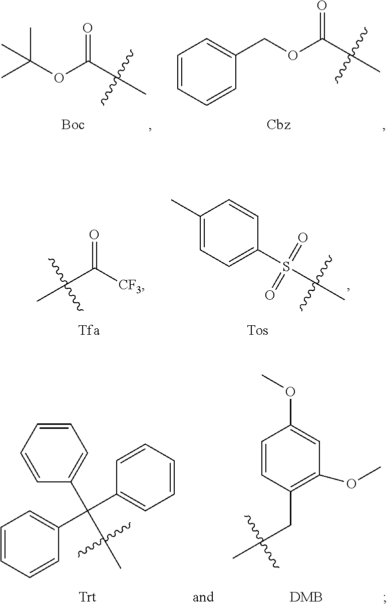

wherein, the first organic solvent is selected from the group consisting of acetonitrile, absolute ethanol, and methanol; and

the second organic solvent is selected from the group consisting of acetonitrile, absolute ethanol, and methanol.

In some specific embodiments, the compound 1 is selected from the group consisting of the following structures:

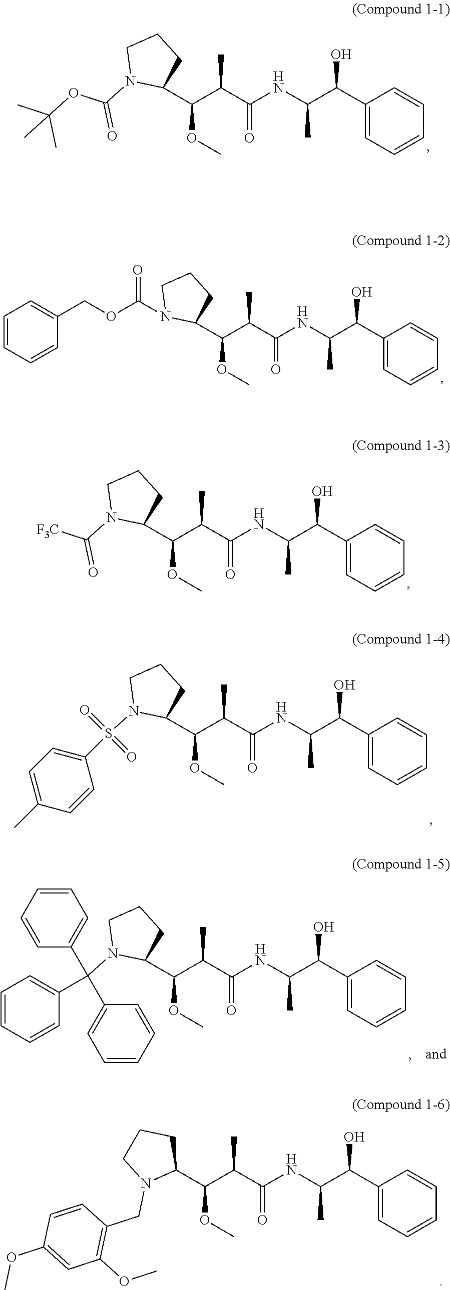 [SMILES: CO[C@H]([C@@H](C)C(=O)N[C@H](C)[C@@H](O)c1ccccc1)[C@@H]1CCCN1C(=O)OC(C)(C)C ; CO[C@H]([C@@H](C)C(=O)N[C@H](C)[C@@H](O)c1ccccc1)[C@@H]1CCCN1C(=O)OCc1ccccc1 ; CO[C@H]([C@@H](C)C(=O)N[C@H](C)[C@@H](O)c1ccccc1)[C@@H]1CCCN1C(=O)C(F)(F)F ; CO[C@H]([C@@H](C)C(=O)N[C@H](C)[C@@H](O)c1ccccc1)[C@@H]1CCCN1S(=O)(=O)c1ccc(C)cc1 ; CO[C@H]([C@@H](C)C(=O)N[C@H](C)[C@@H](O)c1ccccc1)[C@@H]1CCCN1C(c1ccccc1)(c1ccccc1)c1ccccc1 ; COc1ccc(CN2CCC[C@H]2[C@H](OC)[C@@H](C)C(=O)N[C@H](C)[C@@H](O)c2ccccc2)c(OC)c1]

In some more specific embodiments, the structure of the compound 1 is:

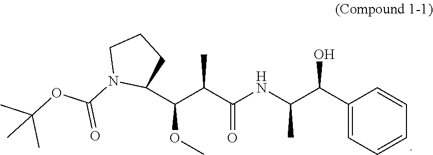 [SMILES: CO[C@H]([C@@H](C)C(=O)N[C@H](C)[C@@H](O)c1ccccc1)[C@@H]1CCCN1C(=O)OC(C)(C)C]

In other more specific embodiments, the structure of the compound 1 is:

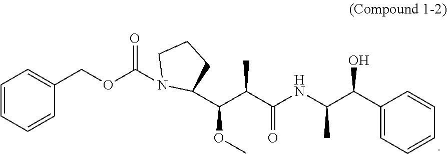 [SMILES: CO[C@H]([C@@H](C)C(=O)N[C@H](C)[C@@H](O)c1ccccc1)[C@@H]1CCCN1C(=O)OCc1ccccc1]

In other more specific embodiments, the structure of the compound 1 is:

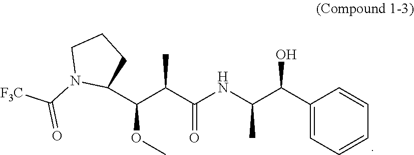 [SMILES: CO[C@H]([C@@H](C)C(=O)N[C@H](C)[C@@H](O)c1ccccc1)[C@@H]1CCCN1C(=O)C(F)(F)F]

In other more specific embodiments, the structure of the compound 1 is:

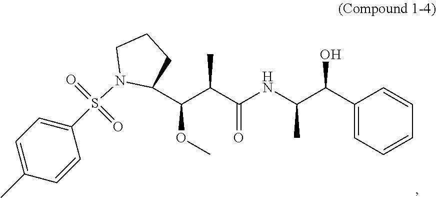 [SMILES: CO[C@H]([C@@H](C)C(=O)N[C@H](C)[C@@H](O)c1ccccc1)[C@@H]1CCCN1S(=O)(=O)c1ccc(C)cc1]

In other more specific embodiments, the structure of the compound 1 is:

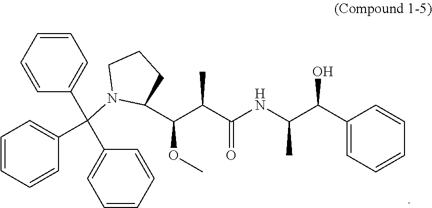 [SMILES: CO[C@H]([C@@H](C)C(=O)N[C@H](C)[C@@H](O)c1ccccc1)[C@@H]1CCCN1C(c1ccccc1)(c1ccccc1)c1ccccc1]

In other more specific embodiments, the structure of the compound 1 is:

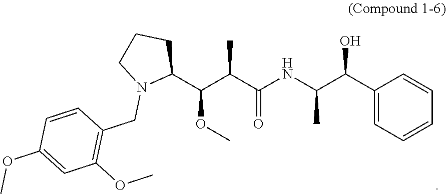 [SMILES: COc1ccc(CN2CCC[C@H]2[C@H](OC)[C@@H](C)C(=O)N[C@H](C)[C@@H](O)c2ccccc2)c(OC)c1]

In some preferred embodiments, the step D further comprises a step D1 comprising: adding an appropriate amount of a third organic solvent to the concentrate b obtained in step C, stirring and dissolving to obtain a solution c; wherein the third organic solvent is selected from the group consisting of methanol and absolute ethanol.

In some other preferred embodiments, the step D further comprises a step D2 performed after the step D1, comprising: adding an appropriate amount of a fourth organic solvent to the solution c obtained in the step D1, filtering, collecting a filter cake, and drying to obtain the compound represented by formula (I); wherein the fourth organic solvent is selected from the group consisting of n-hexane and methyl tert-butyl ether.

In some preferred embodiments, a weight-to-volume ratio (g/ml) of the compound 1 in step A to the first organic solvent in step B is 1:4-15. In some specific embodiments, a weight-to-volume ratio (g/ml) of the compound 1 in step A to the first organic solvent in step B is 1:4, 1:5, 1:6, 1:7, 1:8, 1:9, 1:10, 1:11, 1:12, 1:13, 1:14 or 1:15. In some specific embodiments, a weight-to-volume ratio of the compound 1 in step A to the first organic solvent in step B can also be other ratios within the above-mentioned range of 1:4-15.

In some preferred embodiments, the concentrating under reduced pressure in step B is performed at a temperature of 20-40° C. In some specific embodiments, the concentrating under reduced pressure in step B is performed at a temperature of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C. In other specific embodiments, the concentrating under reduced pressure in step B is performed at other temperatures within the range of 20-40° C.

In some preferred embodiments, a weight-to-volume ratio (g/ml) of the compound 1 in step A to the second organic solvent in step C is 1:4-15. In some specific embodiments, a weight-to-volume ratio (g/ml) of the compound 1 in step A to the second organic solvent in step C is 1:4, 1:5, 1:6, 1:7, 1:8, 1:9, 1:10, 1:11, 1:12, 1:13, 1:14 or 1:15. In some specific embodiments, a weight-to-volume ratio of the compound 1 in step A to the second organic solvent in step C can also be other ratios within the above range.

In some preferred embodiments, the concentrating under reduced pressure in step C is performed at a temperature of 20-40° C. In some specific embodiments, the concentrating under reduced pressure in step C is performed at a temperature of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C. or 40° C. In some specific embodiments, the concentrating under reduced pressure in step C is performed at other temperatures within the range of 20-40° C.

In some preferred embodiments, a weight-to-volume ratio (g/ml) of the compound 1 in step A to the third organic solvent in step D1 is 1:2-8. In some specific embodiments, a weight-to-volume ratio (g/ml) of the compound 1 in step A to the third organic solvent in step D1 is 1:2, 1:3, 1:4, 1:5, 1:6, 1:7 or 1:8. In other specific embodiments, a weight-to-volume ratio of the compound 1 in step A to the third organic solvent described in step D1 can be other ratios within the above range.

In some preferred embodiments, a weight-to-volume ratio (g/ml) of the compound 1 in step A to the fourth organic solvent in step D2 is 1:10-50. In some specific embodiments, a weight-to-volume ratio (g/ml) of the compound 1 in step A to the fourth organic solvent in step D2 is 1:10, 1:15, 1:20, 1:25, 1:30, 1:35, 1:40, 1:45 or 1:50. In other specific embodiments, a weight-to-volume ratio (g/ml) of the compound 1 in step A to the fourth organic solvent in step D2 can also be other ratios within the range, such as 1:11, 1:12, 1:13, 1:14, 1:16, 1:17, 1:18, 1:19, 1:21, and the like.

In some specific embodiments, a combination of the first organic solvent, the second organic solvent, the third organic solvent, and the fourth organic solvent may be selected from the group consisting of the following combinations:

| | The first | The second | The third | |
|---|---|---|---|---|
| | organic | organic | organic | The fourth |
| Combination | solvent | solvent | solvent | organic solvent |
| Combination 1 | Acetonitrile | Acetonitrile | Absolute | n-Hexane |
| | | | ethanol | |
| Combination 2 | Acetonitrile | Acetonitrile | Absolute | Methyl tert-butyl |
| | | | ethanol | ether |
| Combination 3 | Acetonitrile | Acetonitrile | Methanol | n-Hexane |
| Combination 4 | Acetonitrile | Acetonitrile | Methanol | Methyl tert-butyl |
| | | | | ether |
| Combination 5 | Acetonitrile | Absolute | Absolute | n-Hexane |
| | | ethanol | ethanol | |
| Combination 6 | Acetonitrile | Absolute | Absolute | Methyl tert-butyl |
| | | ethanol | ethanol | ether |
| Combination 7 | Acetonitrile | Absolute | Methanol | n-Hexane |
| | | ethanol | | |
| Combination 8 | Acetonitrile | Absolute | Methanol | Methyl tert-butyl |
| | | ethanol | | ether |
| Combination 9 | Acetonitrile | Methanol | Absolute | n-Hexane |
| | | | ethanol | |
| Combination 10 | Acetonitrile | Methanol | Absolute | Methyl tert-butyl |
| | | | ethanol | ether |
| Combination 11 | Acetonitrile | Methanol | Methanol | n-Hexane |
| Combination 12 | Acetonitrile | Methanol | Methanol | Methyl tert-butyl |
| | | | | ether |
| Combination 13 | Absolute | Acetonitrile | Absolute | n-Hexane |
| | ethanol | | ethanol | |
| Combination 14 | Absolute | Acetonitrile | Absolute | Methyl tert-butyl |
| | ethanol | | ethanol | ether |
| Combination 15 | Absolute | Acetonitrile | Methanol | n-Hexane |
| | ethanol | | | |
| Combination 16 | Absolute | Acetonitrile | Methanol | Methyl tert-butyl |
| | ethanol | | | ether |
| Combination 17 | Absolute | Absolute | Absolute | n-Hexane |
| | ethanol | ethanol | ethanol | |
| Combination 18 | Absolute | Absolute | Absolute | Methyl tert-butyl |
| | ethanol | ethanol | ethanol | ether |
| Combination 19 | Absolute | Absolute | Methanol | n-Hexane |
| | ethanol | ethanol | | |
| Combination 20 | Absolute | Absolute | Methanol | Methyl tert-butyl |
| | ethanol | ethanol | | ether |
| Combination 21 | Absolute | Methanol | Absolute | n-Hexane |
| | ethanol | | ethanol | |
| Combination 22 | Absolute | Methanol | Absolute | Methyl tert-butyl |
| | ethanol | | ethanol | ether |
| Combination 23 | Absolute | Methanol | Methanol | n-Hexane |
| | ethanol | | | |
| Combination 24 | Absolute | Methanol | Methanol | Methyl tert-butyl |
| | ethanol | | | ether |
| Combination 25 | Methanol | Acetonitrile | Absolute | n-Hexane |
| | | | ethanol | |
| Combination 26 | Methanol | Acetonitrile | Absolute | Methyl tert-butyl |
| | | | ethanol | ether |
| Combination 27 | Methanol | Acetonitrile | Methanol | n-Hexane |
| Combination 28 | Methanol | Acetonitrile | Methanol | Methyl tert-butyl |
| | | | | ether |
| Combination 29 | Methanol | Absolute | Absolute | n-Hexane |
| | | ethanol | ethanol | |
| Combination 30 | Methanol | Absolute | Absolute | Methyl tert-butyl |
| | | ethanol | ethanol | ether |
| Combination 31 | Methanol | Absolute | Methanol | n-Hexane |
| | | ethanol | | |
| Combination 32 | Methanol | Absolute | Methanol | Methyl tert-butyl |
| | | ethanol | | ether |
| Combination 33 | Methanol | Methanol | Absolute | n-Hexane |
| | | | ethanol | |
| Combination 34 | Methanol | Methanol | Absolute | Methyl tert-butyl |
| | | | ethanol | ether |
| Combination 35 | Methanol | Methanol | Methanol | n-Hexane |
| Combination 36 | Methanol | Methanol | Methanol | Methyl tert-butyl |
| | | | | ether |

In some specific embodiments, the first organic solvent is acetonitrile, the second organic solvent is acetonitrile, the third organic solvent is absolute ethanol, and the fourth organic solvent is n-hexane.

In some other specific embodiments, the first organic solvent is acetonitrile, the second organic solvent is acetonitrile, the third organic solvent is absolute ethanol, and the fourth organic solvent is methyl tert-butyl ether.

In some other specific embodiments, the first organic solvent is acetonitrile, the second organic solvent is acetonitrile, the third organic solvent is methanol, and the fourth organic solvent is n-hexane.

In some other specific embodiments, the first organic solvent is acetonitrile, the second organic solvent is acetonitrile, the third organic solvent is methanol, and the fourth organic solvent is methyl tert-butyl ether.

[In some other specific embodiments, the first organic solvent is acetonitrile, the second organic solvent is absolute ethanol, the third organic solvent is absolute ethanol, and the fourth organic solvent is n-hexane.

In some other specific embodiments, the first organic solvent is acetonitrile, the second organic solvent is absolute ethanol, the third organic solvent is absolute ethanol, and the fourth organic solvent is methyl tert-butyl ether.

In some other specific embodiments, the first organic solvent is acetonitrile, the second organic solvent is absolute ethanol, the third organic solvent is methanol, and the fourth organic solvent is n-hexane.

In some other specific embodiments, the first organic solvent is acetonitrile, the second organic solvent is absolute ethanol, the third organic solvent is methanol, and the fourth organic solvent is methyl tert-butyl ether.

In some other specific embodiments, the first organic solvent is acetonitrile, the second organic solvent is methanol, the third organic solvent is absolute ethanol, and the fourth organic solvent is n-hexane.

In some other specific embodiments, the first organic solvent is acetonitrile, the second organic solvent is methanol, the third organic solvent is absolute ethanol, and the fourth organic solvent is methyl tert-butyl ether.

In some other specific embodiments, the first organic solvent is acetonitrile, the second organic solvent is methanol, the third organic solvent is methanol, and the fourth organic solvent is n-hexane.

In some other specific embodiments, the first organic solvent is acetonitrile, the second organic solvent is methanol, the third organic solvent is methanol, and the fourth organic solvent is methyl tert-butyl ether.

In some other specific embodiments, the first organic solvent is absolute ethanol, the second organic solvent is acetonitrile, the third organic solvent is absolute ethanol, and the fourth organic solvent is n-hexane.

In some other specific embodiments, the first organic solvent is absolute ethanol, the second organic solvent is acetonitrile, the third organic solvent is absolute ethanol, and the fourth organic solvent is methyl tert-butyl ether.

In some other specific embodiments, the first organic solvent is absolute ethanol, the second organic solvent is acetonitrile, the third organic solvent is methanol, and the fourth organic solvent is n-hexane.

In some other specific embodiments, the first organic solvent is absolute ethanol, the second organic solvent is acetonitrile, the third organic solvent is methanol, and the fourth organic solvent is methyl tert-butyl ether.

In some other specific embodiments, the first organic solvent is absolute ethanol, the second organic solvent is absolute ethanol, the third organic solvent is absolute ethanol, and the fourth organic solvent is n-hexane.

In some other specific embodiments, the first organic solvent is absolute ethanol, the second organic solvent is absolute ethanol, the third organic solvent is absolute ethanol, and the fourth organic solvent is methyl tert-butyl ether.

In some other specific embodiments, the first organic solvent is absolute ethanol, the second organic solvent is absolute ethanol, the third organic solvent is methanol, and the fourth organic solvent is n-hexane.

In some other specific embodiments, the first organic solvent is absolute ethanol, the second organic solvent is absolute ethanol, the third organic solvent is methanol, and the fourth organic solvent is methyl tert-butyl ether.

In some other specific embodiments, the first organic solvent is absolute ethanol, the second organic solvent is methanol, the third organic solvent is absolute ethanol, and the fourth organic solvent is n-hexane.

In some other specific embodiments, the first organic solvent is absolute ethanol, the second organic solvent is methanol, the third organic solvent is absolute ethanol, and the fourth organic solvent is methyl tert-butyl ether.

In some other specific embodiments, the first organic solvent is absolute ethanol, the second organic solvent is methanol, the third organic solvent is methanol, and the fourth organic solvent is n-hexane.

In some other specific embodiments, the first organic solvent is absolute ethanol, the second organic solvent is methanol, the third organic solvent is methanol, and the fourth organic solvent is methyl tert-butyl ether.

In some other specific embodiments, the first organic solvent is methanol, the second organic solvent is acetonitrile, the third organic solvent is absolute ethanol, and the fourth organic solvent is n-hexane.

In some other specific embodiments, the first organic solvent is methanol, the second organic solvent is acetonitrile, the third organic solvent is absolute ethanol, and the fourth organic solvent is methyl tert-butyl ether.

In some other specific embodiments, the first organic solvent is methanol, the second organic solvent is acetonitrile, the third organic solvent is methanol, and the fourth organic solvent is n-hexane.

In some other specific embodiments, the first organic solvent is methanol, the second organic solvent is acetonitrile, the third organic solvent is methanol, and the fourth organic solvent is methyl tert-butyl ether.

In some other specific embodiments, the first organic solvent is methanol, the second organic solvent is absolute ethanol, the third organic solvent is absolute ethanol, and the fourth organic solvent is n-hexane.

In some other specific embodiments, the first organic solvent is methanol, the second organic solvent is absolute ethanol, the third organic solvent is absolute ethanol, and the fourth organic solvent is methyl tert-butyl ether.

In some other specific embodiments, the first organic solvent is methanol, the second organic solvent is absolute ethanol, the third organic solvent is methanol, and the fourth organic solvent is n-hexane.

In some other specific embodiments, the first organic solvent is methanol, the second organic solvent is absolute ethanol, the third organic solvent is methanol, and the fourth organic solvent is methyl tert-butyl ether.

In some other specific embodiments, the first organic solvent is methanol, the second organic solvent is methanol, the third organic solvent is absolute ethanol, and the fourth organic solvent is n-hexane.

In some other specific embodiments, the first organic solvent is methanol, the second organic solvent is methanol, the third organic solvent is absolute ethanol, and the fourth organic solvent is methyl tert-butyl ether.

In some other specific embodiments, the first organic solvent is methanol, the second organic solvent is methanol, the third organic solvent is methanol, and the fourth organic solvent is n-hexane.

In some other specific embodiments, the first organic solvent is methanol, the second organic solvent is methanol, the third organic solvent is methanol, and the fourth organic solvent is methyl tert-butyl ether.

In some preferred embodiments, a non-limiting example of the hydrochloric acid method for removing the amino protecting group R in step A is performed as follows: dissolving compound 1 in an appropriate amount of an organic solvent (such as dichloromethane, tetrahydrofuran, 2-methyltetrahydrofuran, etc.), then adding a hydrochloric acid solution at a certain concentration prepared by HCl and an organic solvent (such as ethyl acetate, ethanol, etc.) or water (wherein the concentration of the hydrochloric acid solution may be 20% to 38%, such as 20%, 21%, 22%, 23%, 24%, 25%, 26%, 27%, 28%, 29%, 30%, 31%, 32%, 33%, 34%, 35%, 36%, 37%, and 38%), and reacting at 10-30° C., wherein a weight-to-volume ratio (g/ml) of compound 1 to the hydrochloric acid solution is 1:1 to 5 (such as 1:1, 1:2, 1:3, 1:4, 1:5 or other ratios within the range).

In some specific embodiments, the hydrochloric acid solution is a solution prepared by mixing concentrated hydrochloric acid with ethanol. In some other specific embodiments, the hydrochloric acid solution is a solution prepared by mixing concentrated hydrochloric acid with water.

In some specific embodiments, the concentration of the hydrochloric acid solution is 25%; in some other specific embodiments, the concentration of the hydrochloric acid solution is 26%; in some other specific embodiments, the concentration of the hydrochloric acid solution is 27%; in some other specific embodiments, the concentration of the hydrochloric acid solution is 28%; in some other specific embodiments, the concentration of the hydrochloric acid solution is 29%; in some other specific embodiments, the concentration of the hydrochloric acid solution is 30%; in some other specific embodiments, the concentration of the hydrochloric acid solution is 31%; in some other specific embodiments, the concentration of the hydrochloric acid solution is 32%; in some other specific embodiments, the concentration of the hydrochloric acid solution is 33%; in some other specific embodiments, the concentration of the hydrochloric acid solution is 34%; in some other specific embodiments, the concentration of the hydrochloric acid solution is 35%; in some other specific embodiments, the concentration of the hydrochloric acid solution is 36%; in some other specific embodiments, the concentration of the hydrochloric acid solution is 37%; and in some other specific embodiments, the concentration of the hydrochloric acid solution is 38%.

In some specific embodiments, the weight-to-volume ratio (g/ml) of the compound 1 to the hydrochloric acid solution is 1:2; in some other specific embodiments, the weight-to-volume ratio (g/ml) of the compound 1 to the hydrochloric acid solution is 1:3; in some other specific embodiments, the weight-to-volume ratio (g/ml) of the compound 1 to the hydrochloric acid solution is 1:4; in some other specific embodiments, the weight-to-volume ratio (g/ml) of the compound 1 to the hydrochloric acid solution is 1:5.

In some more specific embodiments, the step of removing the amino protecting group R by hydrochloric acid method in step A is performed by dissolving the compound 1 in an appropriate amount of dichloromethane, adding a 30% hydrogen chloride solution in ethanol, and reacting at 10-30° C., wherein the weight-to-volume ratio (g/ml) of the compound 1 to the hydrochloric acid solution is 1:3.

In some more specific embodiments, the step of removing the amino protecting group R by hydrochloric acid method in step A is performed by dissolving compound 1 in an appropriate amount of dichloromethane, adding an aqueous solution of 30% hydrogen chloride, and reacting at 10-30° C., wherein the weight volume ratio (g/ml) of the compound 1 to the hydrochloric acid solution is 1:3.

The method for preparing and purifying the monomethyl auristatin E intermediate provided by the present disclosure has few steps in the synthesis method and is simple to operate. In addition, the raw materials are cheap and readily available with high safety. The purification method is simple, and the purified product has extremely high purity up to 91% or more. Moreover, the method provided by the present disclosure can achieve a yield as high as 95%. By using an organic solvent such as acetonitrile, absolute ethanol or methanol to dry the reaction product under reduced pressure, the types of residual impurities in the prepared product are greatly reduced. The method of the present disclosure not only improves the safety of the production process, but also significantly improves the yield and purity of the product, and reduces the subsequent production cost, which is suitable for industrial production.

### BRIEF DESCRIPTION OF DRAWINGS

FIG. 1 is a chromatogram of the compound represented by formula (I) prepared by the preparation method of Example 1;

FIG. 2 is a chromatogram of the compound represented by formula (I) prepared by the preparation method of Example 2;

FIG. 3 is a chromatogram of the compound represented by formula (I) prepared by the preparation method of Example 3;

FIG. 4 is a chromatogram of the compound represented by formula (I) prepared by the preparation method of Example 4;

FIG. 5 is a chromatogram of the compound represented by formula (I) prepared by the preparation method of Example 5;

FIG. 6 is a chromatogram of the compound represented by formula (I) prepared by the preparation method of Example 6;

FIG. 7 is a chromatogram of the compound represented by formula (I) prepared by the preparation method of Example 7;

FIG. 8 is a chromatogram of the compound represented by formula (I) prepared by the preparation method of Comparative example 1;

FIG. 9 is a chromatogram of the compound represented by formula (I) prepared by the preparation method of Comparative example 2;

FIG. 10 is a chromatogram of the compound represented by formula (I) prepared by the preparation method of Comparative example 3; and

FIG. 11 is a chromatogram of the compound represented by formula (I) prepared by the preparation method of Comparative example 4.

### DETAILED DESCRIPTION

The technical solution of the present disclosure is further described in detail in a non-limiting way in conjunction with the specific examples. It should be pointed out that the following examples are only for illustrating the technical concept and features of the present disclosure. Their purpose is to enable those skilled in the art to understand the content of the present disclosure and implement it accordingly, and they cannot limit the scope of protection of the present disclosure. All equivalent changes or modifications made according to the spirit of the present disclosure should be included in the scope of protection of the present disclosure.

### EXAMPLE 1

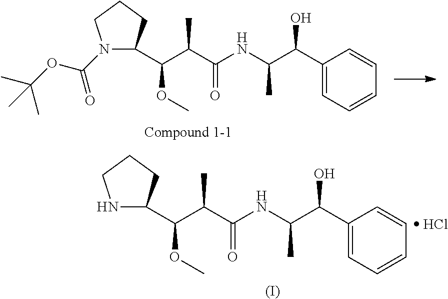

Boc protecting group in compound 1-1 was removed by hydrochloric acid method. The specific steps involved in this example were as follows: 500 mg of compound 1-1 and 1.5 ml of dichloromethane were added to a three-necked flask and stirred until dissolved, then 1.5 ml of a solution of 30% hydrogen chloride in ethanol was added dropwise, and the reaction was performed at a temperature controlled within 10-30° C. for 2 hours.

Afterwards, 3 ml of acetonitrile was added to the above reaction solution and stirred evenly, and then the mixture was transferred to a single-necked flask and concentrated under reduced pressure in a water bath at 24-28° C.

After the concentration was completed, 5 ml of acetonitrile was added to the single-necked bottle again. The mixture was shaken evenly and then concentrated to dryness under reduced pressure in a water bath at 24-28° C.

The concentrated product obtained in the previous step was added with 1.5 ml of absolute ethanol and stirred to dissolve, and then 7.5 ml of methyl tert-butyl ether was slowly added dropwise. A large amount of white solid was precipitated in the bottle. The mixture was subjected to suction filtration. The filter cake was collected and dried under vacuum at 20-30° C. for not less than 12 hours until the weight no longer changed, to obtain 403 mg of the compound represented by formula (I), with a yield of 95%, a purity of 99.79%, and a maximum single impurity of 0.14% (see FIG. 1).

### EXAMPLE 2

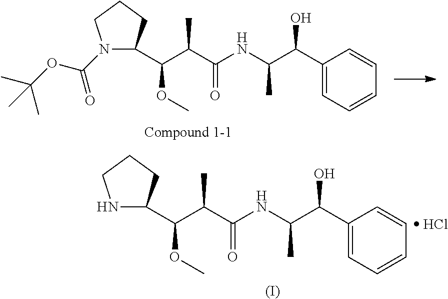

Boc protecting group in compound 1-1 was removed by hydrochloric acid method. The specific steps involved in this example were as follows: 800 mg of compound 1-1 and 4 ml of tetrahydrofuran were added to a three-necked bottle and stirred until dissolved, then 4 ml of a solution of 25% hydrogen chloride in ethanol was added dropwise, and the reaction was performed at a temperature controlled within 20-25° C. for 2 hours.

Afterwards, 6.4 ml of absolute ethanol was added to the above reaction solution and stirred evenly, and then the mixture was transferred to a single-necked bottle and concentrated under reduced pressure in a water bath at 24-28° C.

After the concentration was completed, 6.4 ml of absolute ethanol was added to the single-necked bottle again. The mixture was shaken evenly, and then concentrated to dryness under reduced pressure in a water bath at 24-28° C.

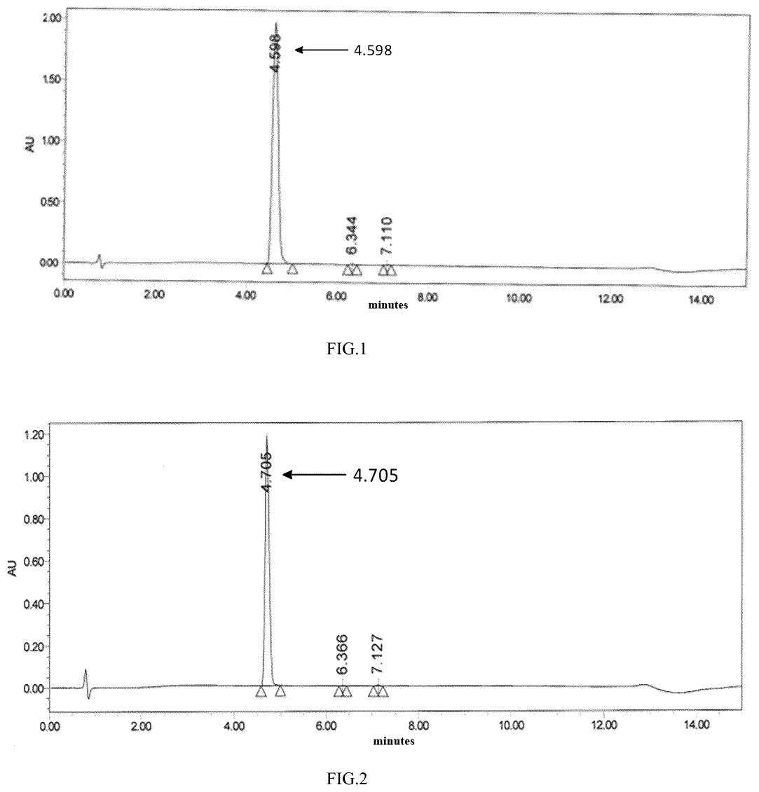

The concentrated product obtained in the previous step was added with 4 ml of absolute ethanol and stirred to dissolve, and then 20 ml of methyl tert-butyl ether was slowly added dropwise. A large amount of white solid was precipitated in the bottle. The mixture was subjected to suction filtration. The filter cake was collected and dried under vacuum at 20-30° C. for not less than 12 hours until the weight no longer changed, to obtain 642 mg of the compound represented by formula (I), with a yield of 94.5%, a purity of 99.53%, and a maximum single impurity of 0.30% (see FIG. 2).

### EXAMPLE 3

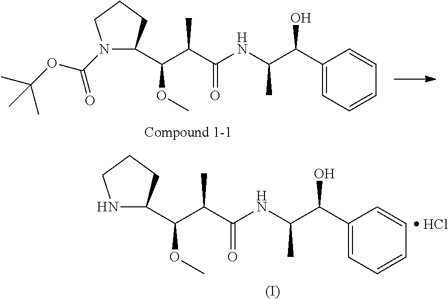

Boc protecting group in compound 1-1 was removed by hydrochloric acid method. The specific steps involved in this example were as follows: 600 mg of compound 1-1 and 3 ml of dimethyltetrahydrofuran were added to a three-necked flask and stirred until dissolved. Then 2.4ml of an aqueous solution of 36% hydrogen chloride was added dropwise. The reaction was performed at a temperature controlled within 15-20° C. for 3 hours.

6 ml of methanol was added to the above reaction solution and stirred evenly. Afterwards, the mixed solution was transferred to a single-necked flask and concentrated under reduced pressure in a water bath at 24-28° C.

After the concentration was completed, 4.8 ml of acetonitrile was added to the single-necked flask again and stirred evenly. Then the mixed solution was concentrated to dryness under reduced pressure in a water bath at 24-28° C.

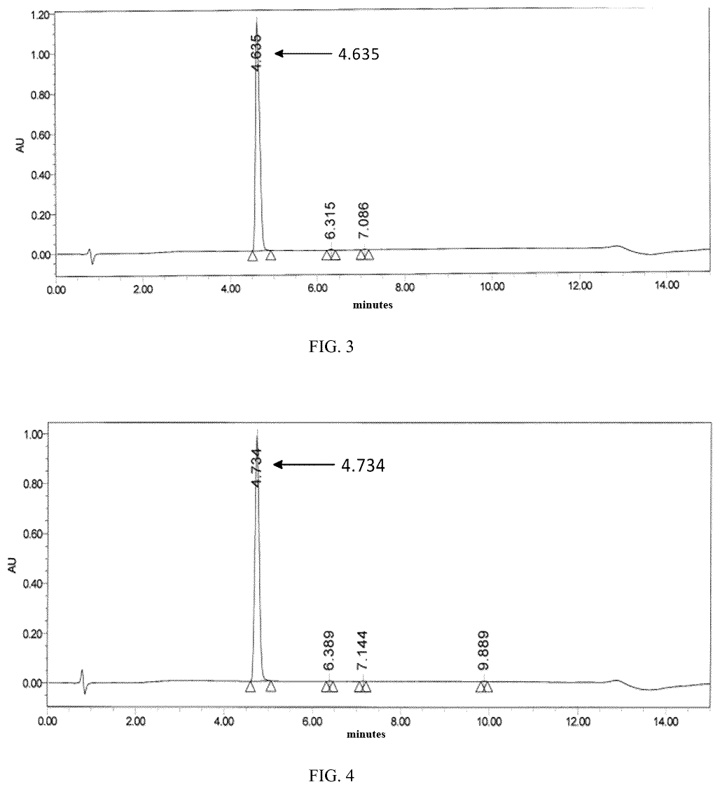

The concentrated product obtained in the previous step was added with 2.4 ml of methanol and stirred to dissolve. Then 12 ml of n-hexane was slowly added dropwise. A large amount of white solid was precipitated in the bottle. The mixture was subjected to suction filtration. The filter cake was collected and dried under vacuum at 30-35° C. for not less than 12 hours until the weight no longer changed, to obtain 475 mg of the compound represented by formula (I), with a yield of 93.2%, a purity of 99.43%, and a maximum single impurity of 0.37% (see FIG. 3).

### EXAMPLE 4

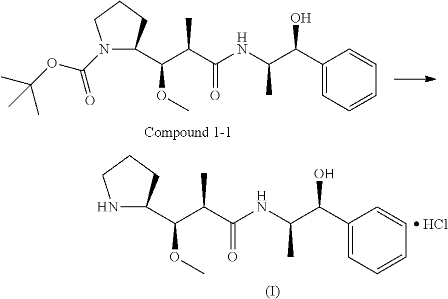

Boc protecting group in compound 1-1 was removed by hydrochloric acid method. The specific steps involved in this example were as follows: 10.0 g of compound 1-1 and 60 ml of dichloromethane were added to a three-necked flask and stirred until dissolved. Then 40 ml of a solution of 25% hydrogen chloride in methanol was added dropwise. The reaction was performed at a temperature within 15-20° C. for 3 hours.

100 ml of methanol was added to the reaction solution, and stirred evenly. Then the mixture was transferred to a single-necked flask, and concentrated under reduced pressure in a water bath at 26° C.

After the concentration was completed, 100 ml of methanol was added to the single-necked bottle. The mixture was shaken evenly, and concentrated to dryness under reduced pressure in a 26° C. water bath. The same volume of methanol was added repeatedly and then dried under reduced pressure once.

The concentrated product obtained in the previous step was added with 30 ml of methanol, and stirred to dissolve. Then 150 ml of n-hexane was slowly added dropwise. A large amount of white solid was precipitated in the bottle. The mixture was subjected to suction filtration. The filter cake was collected and dried under vacuum at 30-35° C. for not less than 12 hours until the weight no longer changed, to obtain 8.0 g of the compound represented by formula (I), with a yield of 94.2%, a purity of 99.62%, and a single impurity of 0.13% (see FIG. 4).

### EXAMPLE 5

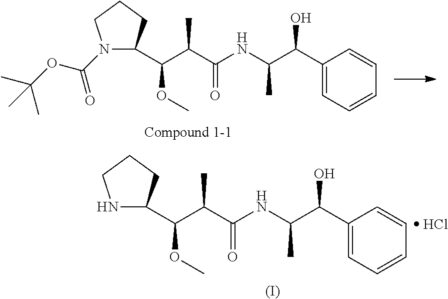

Boc protecting group in compound 1-1 was removed by hydrochloric acid method. The specific steps involved in this example were as follows: 500 mg of compound 1-1 and 2 ml of dichloromethane were added to a three-necked bottle, and stirred until dissolved. Then 1.5 ml of a solution of 32% hydrogen chloride in ethanol was added dropwise. The reaction was performed at a temperature controlled within 10-20° C. for 3 hours.

Then, 2 ml of acetonitrile was added to the above reaction solution and stirred evenly. The mixture was transferred to a single-necked bottle and concentrated under reduced pressure in a water bath at 24-28° C.

After the concentration was completed, 2 ml of acetonitrile was added to the single-necked bottle again. The mixture was shaken evenly and concentrated to dryness under reduced pressure in a water bath at 24-28° C.

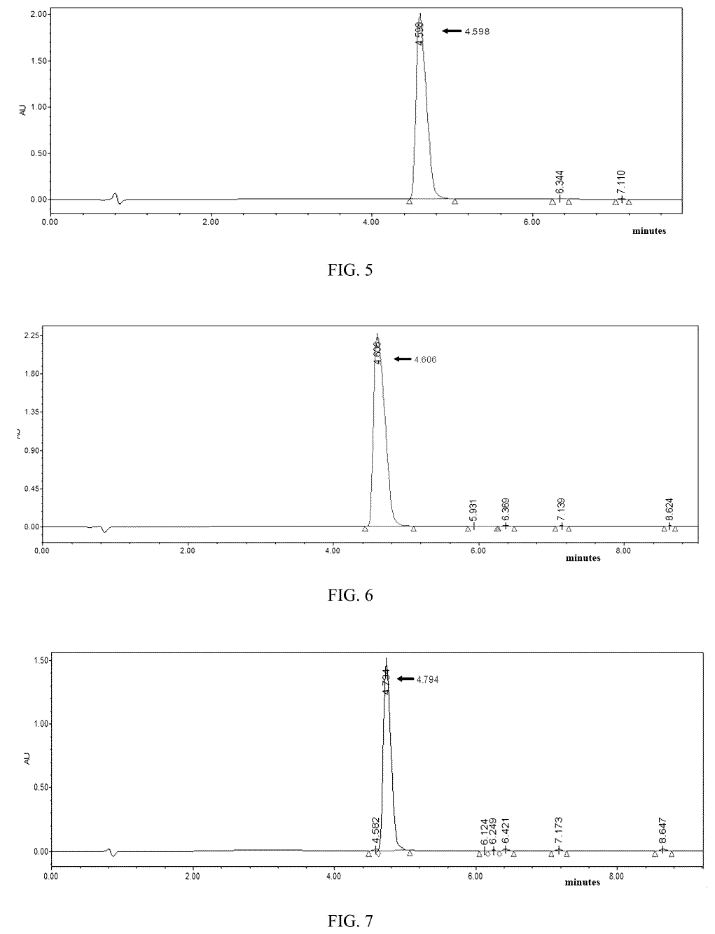

The concentrated product obtained in the previous step was added with 1 ml of absolute ethanol, and stirred to dissolve. Then 5 ml of methyl tert-butyl ether was slowly added dropwise. A large amount of white solid was precipitated in the bottle. The mixture was subjected to suction filtration. The filter cake was collected and dried under vacuum at 20-30° C. for not less than 12hours until the weight no longer changed, to obtain 386 mg of the compound represented by formula (I), with a yield of 91%, a purity of 99.63%, and a maximum single impurity of 0.16% (see FIG. 5).

### EXAMPLE 6

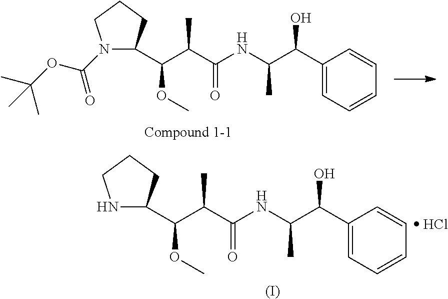

Boc protecting group in compound 1-1 was removed by hydrochloric acid method. The specific steps involved in this example were as follows: 1 g of compound 1-1 and 4 ml of tetrahydrofuran were added to a three-necked flask and stirred until dissolved. Then 4 ml of a solution of 27% hydrogen chloride in ethanol was added dropwise. The reaction was performed at a temperature controlled within 20-30° C. for 2 hours.

15 ml of acetonitrile was then added to the above reaction solution and stirred evenly. The mixture was then transferred to a single-necked flask and concentrated under reduced pressure in a water bath at 24-28° C.

After the concentration was completed, 15 ml of acetonitrile was added to the single-necked flask again. The mixture was shaken evenly and concentrated to dryness under reduced pressure in a water bath at 24-28° C.

The concentrated product obtained in the previous step was added with 8 ml of absolute

ethanol. Then 50 ml of methyl tert-butyl ether was slowly added dropwise. A large amount of white solid was precipitated in the bottle. The mixture was subjected to suction filtration. The filter cake was collected and dried under vacuum at 20-30° C. for not less than 12 h until the weight no longer changed, to obtain 789 mg of the compound represented by formula (I), with a yield of 93%, a purity of 99.53%, and a maximum single impurity of 0.17% (see FIG. 6).

### EXAMPLE 7

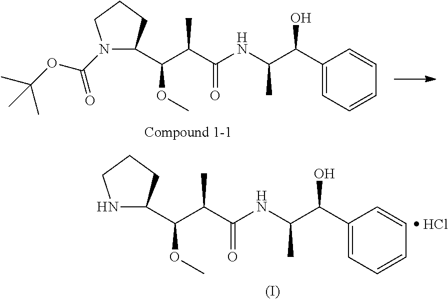

Boc protecting group in compound 1-1 was removed by hydrochloric acid method. The specific steps involved in this example were as follows: 2 g of compound 1-1 and 4 ml of tetrahydrofuran were added to a three-necked flask and stirred until dissolved, and then 4 ml of a solution of 36% hydrogen chloride in ethanol was added dropwise. The reaction was performed at a temperature controlled within 10-30° C. for 2 h.

Then 14 ml of methanol was added to the above reaction solution and stirred evenly. The mixture was then transferred to a single-necked flask and concentrated under reduced pressure in a water bath at 24-28° C.

After the concentration was completed, 14 ml of methanol was added to the single-necked flask again. The mixture was shaken evenly and then concentrated to dryness under reduced pressure in a water bath at 24-28° C.

The concentrated product obtained in the previous step was added with 8 ml of absolute ethanol, and stirred to dissolve. Then 20 ml of methyl tert-butyl ether was slowly added dropwise. A large amount of white solid was precipitated in the flask. The mixture was subjected to suction filtration. The filter cake was collected and dried under vacuum at 20-30° C. for not less than 12 h until the weight no longer changed, to obtain 1561 mg of the compound represented by formula (I), with a yield of 92%, a purity of 99.32%, and a maximum single impurity of 0.23% (see FIG. 7).

### Comparative Example 1

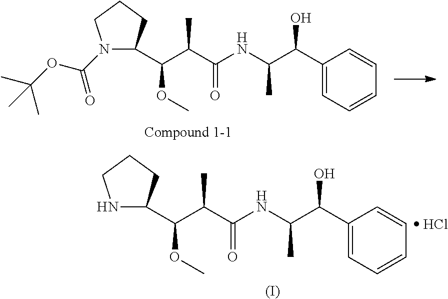

5.0 g of compound 1-1 and 10 ml of dichloromethane were added to a three-necked flask and stirred until dissolved. Then 20 ml of a solution of 25% hydrogen chloride in ethanol was added dropwise. The reaction was performed at 15-20° C. for 1 h.

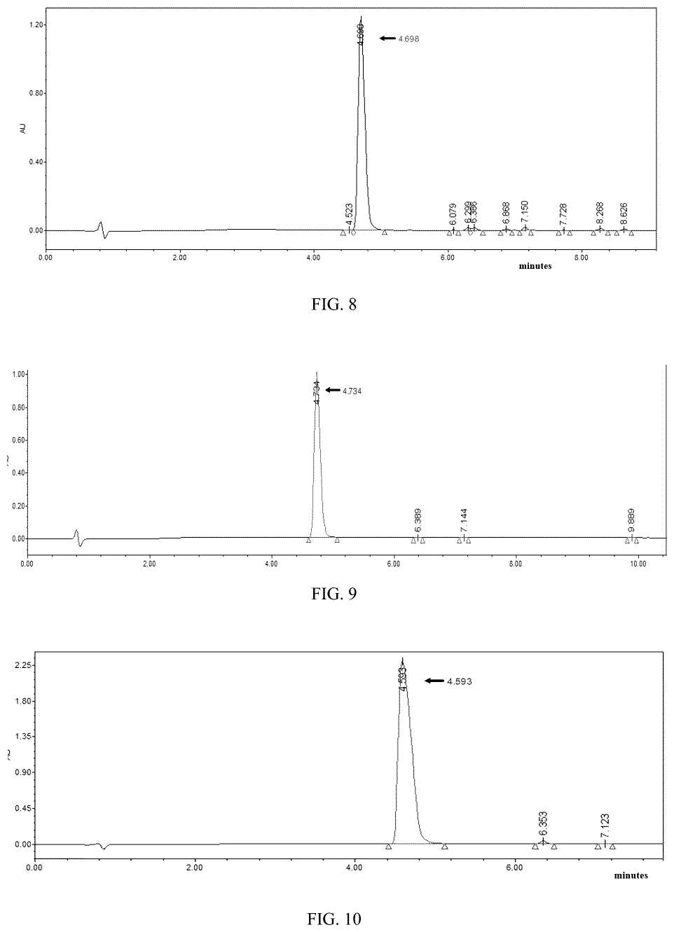

The reaction solution was poured into 80 ml of n-hexane at 0-5° C. and stirred to precipitate an oily substance. The supernatant was discarded, and the residue was dried under vacuum until the weight no longer changed, to obtain 3.81 g of the compound represented by formula (I), with a yield of 89.8%, a purity of 94.6%, and a maximum single impurity of 1.40% (see FIG. 8)

### Comparative Example 2

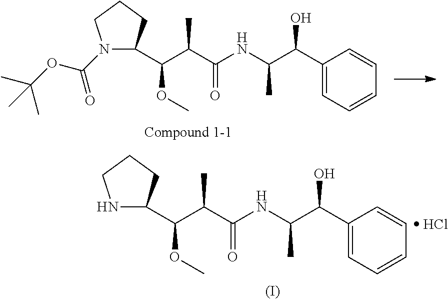

Boc protecting group in compound 1-1 was removed by hydrochloric acid method. The specific steps involved in this example were as follows: 100 mg of compound 1-1 and 0.5 ml of dichloromethane were added to a three-necked flask and stirred until dissolved. Then 0.5 ml of a solution of 32% hydrogen chloride in ethanol was added dropwise. The reaction was performed at a temperature controlled within 20-30° C. for 1 hour.

Afterwards, 0.5 ml of acetonitrile was added to the above reaction solution and stirred evenly. Then the mixture was transferred to a single-necked flask and concentrated to dryness under reduced pressure in a water bath at 24-28° C.

The concentrated product obtained in the previous step was added with 0.5 ml of absolute

ethanol and stirred to dissolve. Then 5 ml of methyl tert-butyl ether was slowly added dropwise. A large amount of white solid was precipitated in the bottle. The mixture was subjected to suction filtration. The filter cake was collected and dried under vacuum at 20-30° C. for not less than 12 hours until the weight no longer changed, to obtain 71 mg of the compound represented by formula (I), with a yield of 74%, a purity of 99.54%, and a maximum single impurity of 0.13% (see FIG. 9).

### Comparative Example 3

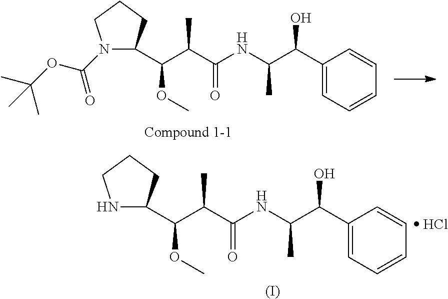

Boc protecting group in compound 1-1 was removed by hydrochloric acid method. The specific steps involved in this example were as follows: 1 g of compound 1-1 and 6 ml of dichloromethane were added to a three-necked flask and stirred until dissolved. Then 4 ml of a solution of 27% hydrogen chloride in ethanol was added dropwise. The reaction was performed at 15-20° C. for 3 hours.

10 ml of 1,4-dioxane was added to the reaction solution, and stirred evenly. Then the mixture was transferred to a single-necked flask and concentrated under reduced pressure in a water bath at 24-28° C.

After the concentration was completed, 10 ml of 1,4-dioxane was added to the single-necked bottle. The mixture was shaken well, and concentrated to dryness under reduced pressure in a water bath at 24-28° C.

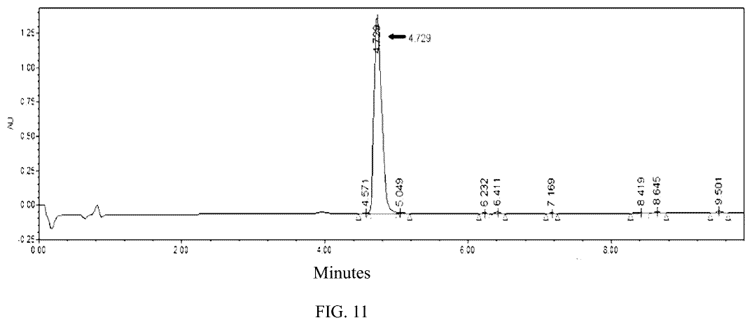

The concentrated product obtained in the previous step was added with 5 ml of methanol, and stirred to dissolve. Then 15 ml of methyl tert-butyl ether was slowly added dropwise. A small amount of white solid was precipitated in the bottle. The mixture was subjected to suction filtration. The filter cake was collected and dried under vacuum at 30-35° C. for not less than 12 hours until the weight no longer changed, to obtain 0.5 g of the compound represented by formula (I), with a yield of 58.9%, a purity of 99.57%, and a maximum single impurity of 0.19% (see FIG. 10).

### Comparative Example 4

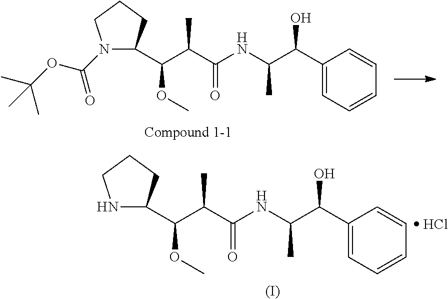

Boc protecting group in compound 1-1 was removed by hydrochloric acid method. The specific steps involved in this example were as follows: 600 mg of compound 1-1 and 2.4 ml of dichloromethane were added to a three-necked flask and stirred until dissolved. Then, 1.8 ml of a solution of 30% hydrogen chloride in ethanol was added dropwise. The reaction was performed at a temperature controlled within 10-20° C. for 3 hours.

Afterwards, 3 ml of acetonitrile was added to the above reaction solution and stirred evenly. Then, the mixed solution was transferred to a single-necked flask and concentrated under reduced pressure in a water bath at 24-28° C.

After the concentration was completed, 3 ml of acetonitrile was added to the single-necked flask again. The mixed solution was shaken evenly and concentrated to dryness under reduced pressure in a water bath at 24-28° C.

The concentrated product obtained in the previous step was added with 3 ml of dichloromethane, and stirred to dissolve. Then 6 ml of n-heptane was slowly added dropwise. A white solid was precipitated in the bottle. The mixture was subjected to suction filtration. The filter cake was collected and dried under vacuum at 20-30° C. for not less than 12 hours until the weight no longer changed, to obtain 342 mg of the compound represented by formula (I), with a yield of 67.1%, a purity of 99.02%, and a maximum single impurity of 0.35% (see FIG. 11).

| Example | Yield | Purity | Single impurity |
|---|---|---|---|
| Example 1 | 95.00% | 99.79% | 0.14% |
| Example 2 | 94.50% | 99.53% | 0.30% |
| Example 3 | 93.20% | 99.43% | 0.37% |
| Example 4 | 94.20% | 99.62% | 0.13% |
| Example 5 | 91.00% | 99.63% | 0.16% |
| Example 6 | 93.00% | 99.53% | 0.17% |
| Example 7 | 92.00% | 99.32% | 0.23% |
| Comparative example 1 | 89.80% | 94.60% | 1.40% |
| Comparative example 2 | 74.00% | 99.54% | 0.13% |
| Comparative example 3 | 58.90% | 99.57% | 0.19% |
| Comparative example 4 | 67.10% | 99.02% | 0.35% |

By comparison, the method for preparing and purifying the monomethyl auristatin E intermediate provided by the present disclosure has few steps in the synthesis method and is easy to operate. In addition, the raw materials are cheap and readily available with high safety. The purification method is simple, and the purified product has extremely high purity up to 91% or more. Moreover, the method provided by the present disclosure can achieve a yield as high as 95%. By using an organic solvent such as acetonitrile, absolute ethanol or methanol to dry the reaction product under reduced pressure, the types of residual impurities in the prepared product are greatly reduced. The method of the present disclosure not only improves the safety of the production process, but also significantly improves the yield and purity of the product, and reduces the subsequent production cost, which is suitable for industrial production.

The present disclosure has been illustrated by various specific examples. However, it can be understood by those of ordinary skill in the art that the present disclosure is not limited to various specific embodiments, and those of ordinary skill in the art can make various changes or modifications within the scope of the present disclosure, and the various technical features mentioned in various places in this specification can be combined with each other without departing from the spirit and scope of the present disclosure. Such changes and modifications are all within the scope of the present disclosure.

1. A method for preparing and purifying a monomethyl auristatin E intermediate, wherein the monomethyl auristatin E intermediate has a structure represented by formula (I): 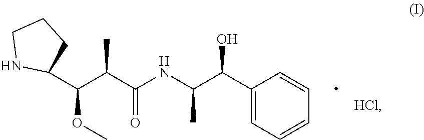 [SMILES: CO[C@@H]([C@@H]1CCCN1)[C@@H](C)C(=O)N[C@H](C)[C@@H](O)c1ccccc1]
the method has a preparation route of: 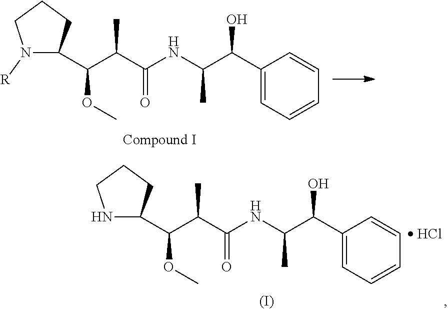
wherein R is an amino protecting group, and the method comprises the following steps:
A. removing the amino protecting group R from compound 1 by hydrochloric acid method;
B. after completing the reaction of step A, adding an appropriate amount of a first organic solvent to a reaction solution of step A, and concentrating under reduced pressure to obtain a concentrate a;
C. adding an appropriate amount of a second organic solvent to the concentrate a obtained in step B, and concentrating under reduced pressure to obtain a concentrate b; and
D. further purifying the concentrate b,
wherein:
the amino protecting group R is selected from the group consisting of Boc protecting group, Cbz protecting group, Tfa protecting group, Tos protecting group, Trt protecting group, and DMB protecting group;
the first organic solvent is selected from the group consisting of acetonitrile, methanol and absolute ethanol; and
the second organic solvent is selected from the group consisting of acetonitrile, methanol and absolute ethanol.

2. The method according to claim 1, wherein the step D further comprises a step D1 comprising: adding an appropriate amount of a third organic solvent to the concentrate b obtained in step C, stirring and dissolving to obtain a solution c; wherein the third organic solvent is selected from the group consisting of methanol and absolute ethanol.

3. The method according to claim 2, wherein the step D further comprises a step D2 performed after the step D1, comprising: adding an appropriate amount of a fourth organic solvent to the solution c obtained in the step D1, filtering, collecting a filter cake, and drying to obtain the compound represented by formula (I); wherein the fourth organic solvent is selected from the group consisting of n-hexane and methyl tert-butyl ether.

4. The method according to claim 1, wherein the compound 1 is selected from the group consisting of the following structures:

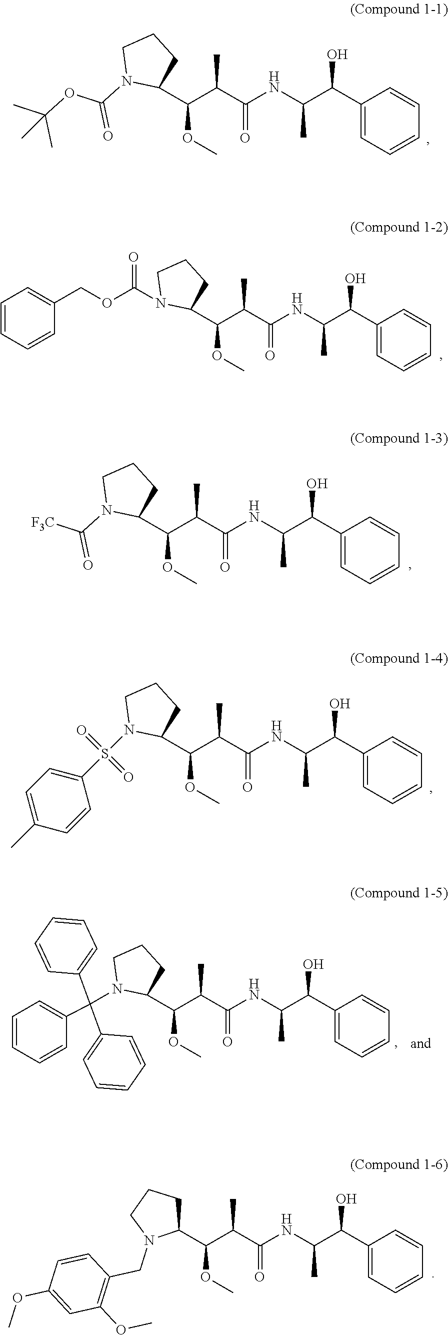 [SMILES: CO[C@H]([C@@H](C)C(=O)N[C@H](C)[C@@H](O)c1ccccc1)[C@@H]1CCCN1C(=O)OC(C)(C)C ; CO[C@H]([C@@H](C)C(=O)N[C@H](C)[C@@H](O)c1ccccc1)[C@@H]1CCCN1C(=O)OCc1ccccc1 ; CO[C@H]([C@@H](C)C(=O)N[C@H](C)[C@@H](O)c1ccccc1)[C@@H]1CCCN1C(=O)C(F)(F)F ; CO[C@H]([C@@H](C)C(=O)N[C@H](C)[C@@H](O)c1ccccc1)[C@@H]1CCCN1S(=O)(=O)c1ccc(C)cc1 ; CO[C@H]([C@@H](C)C(=O)N[C@H](C)[C@@H](O)c1ccccc1)[C@@H]1CCCN1C(c1ccccc1)(c1ccccc1)c1ccccc1 ; COc1ccc(CN2CCC[C@H]2[C@H](OC)[C@@H](C)C(=O)N[C@H](C)[C@@H](O)c2ccccc2)c(OC)c1]

5. The method according to claim 1, wherein a weight-to-volume ratio (g/ml) of the compound 1 in step A to the first organic solvent in step B is 1:4-15.

6. The method according to claim 1, wherein the concentrating under reduced pressure in step B is performed at a temperature of 20-40° C.

7. The method according to claim 1, wherein a weight-to-volume ratio (g/ml) of the compound 1 in step A to the second organic solvent in step C is 1:4-15.

8. The method according to claim 1, wherein the concentrating under reduced pressure in step C is performed at a temperature of 20-40° C.

9. The method according to claim 1, wherein a weight-to-volume ratio (g/ml) of the compound 1 in step A to the third organic solvent in step D1 is 1:2-8.

10. The method according to claim 1, wherein a weight-to-volume ratio (g/ml) of the compound 1 in step A to the fourth organic solvent in step D2 is 1:10-50.

11. A method to remove reaction impurities in the preparation of a monomethyl auristatin E intermediate, wherein the monomethyl auristatin E intermediate has a structure represented by formula (I): 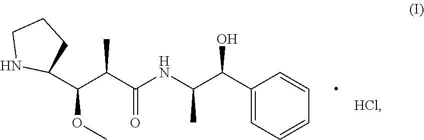 [SMILES: CO[C@@H]([C@@H]1CCCN1)[C@@H](C)C(=O)N[C@H](C)[C@@H](O)c1ccccc1]
the preparation has a route of: 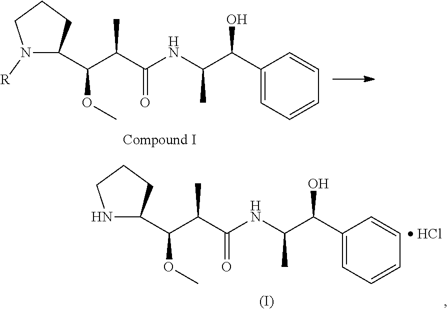
wherein R is an amino protecting group, and the method comprises concentrating the product prepared by the above route under reduced pressure using acetonitrile, absolute ethanol or methanol.
